(19) United States
(12) Patent Application Publication
(10) Pub. No.: US 2024/0141029 A1
(43) Pub. Date: May 2, 2024

(54) METHODS AND COMPOSITIONS FOR TREATING SEPSIS

(71) Applicant: Anji Pharmaceuticals Inc., Cambridge, MA (US)

(72) Inventors: Brian K. Hubbard, Boxford, MA (US); Michael H. Serrano-Wu, Belmont, MA (US); Charles D. Meyers, Littleton, MA (US)

(21) Appl. No.: 18/279,671

(22) PCT Filed: Mar. 2, 2022
(86) PCT No.: PCT/US22/18507

§ 371 Date: Aug. 31, 2023

(51) Int. Cl. C07K 16/22; C07K 16/40

(52) U.S. Cl. CPC C07K 16/22; C07K 16/40; A61K 2039/507

(57) ABSTRACT

Provided herein are methods and compositions related to the treatment of sepsis using anti-ANGPTL3 antibodies.

### CROSS-REFERENCE TO RELATED APPLICATION

This application claims the benefit of U.S. Provisional Application Ser. No. 63/157,339, filed Mar. 5, 2021, the entire contents of which are incorporated herein by reference.

### BACKGROUND

Sepsis arises from a host response to an infection caused by bacteria or other infectious agents such as viruses, fungi, and parasites in the bloodstream. Typically, when sepsis arises, the host is unable to break down clots that are formed in the lining of inflamed blood vessels, limiting blood flow to the organs and subsequently leading to organ failure or gangrene.

Sepsis generally begins with infection, followed by systemic inflammatory response syndrome (SIRS), followed by severe sepsis, and finally septic shock, which causes multiple organ dysfunction and death. Worldwide incidence of sepsis continues to rise, with increasing concern for elderly patients due to an aging population. Approximately one-third to one-half of all severe sepsis patients succumb to their illness. Effective, cost-efficient therapies for preventing or treating sepsis are urgently needed.

### SUMMARY

The present invention is based on the surprising discovery that anti-ANGPTL3 antibodies, which are currently used to reduce LDL cholesterol levels, can facilitate the neutralization of microbial toxins and/or clear such toxins from the bloodstream. The compositions and methods of the present invention can be used for the treatment of sepsis as well as neutralizing bacterial toxins in a subject.

The compositions and methods described herein can be used to prevent or treat sepsis in a subject by administering an antibody that specifically binds ANGPTL3 to a subject in need.

Further, the compositions and methods described herein can be used to prevent or treat organ tissue damage in a subject. The method comprises administering an antibody that specifically binds ANGPTL3 to the subject. The organ tissue can be liver, kidney, central nervous system tissue, and any other tissue that can be impacted by sepsis or sepsis-like conditions.

This invention also provides methods to modulate an immune response in a subject having or suspected of having sepsis. This method comprises administering to the subject an antibody that specifically binds ANGPTL3.

The compositions and methods described herein can be used to treat any stage of sepsis or septic condition. For example, systemic inflammatory response syndrome (SIRS), severe sepsis, and/or septic shock can be treated using the methods and compositions described herein.

Antibodies that specifically bind ANGPTL3 can be included in the compositions or used in the methods described. The antibody can be monoclonal or polyclonal. Further, the antibody can be a chimeric and/or humanized antibody. An example of an antibody that specifically binds ANGPTL3 is evinacumab.

Delivery of an antibody that specifically binds ANGPTL3 can be by any delivery method, including but not limited to intravenously (e.g., intravenous infusion), intraperitoneally, intramuscularly, intraarterially, intraportally, intralesionally, orally, and subcutaneously. In some embodiments, the antibody that specifically binds ANGPTL3 is administered continuously via intravenous infusion.

A therapeutically effective dose of a composition comprising an antibody that specifically binds ANGPTL3 is about 5 mg, about 10 mg, about 50 mg, about 100 mg, about 150 mg, about 200 mg, about 250 mg, about 300 mg, about 350 mg, about 400 mg, or about 450 mg. The antibody that specifically binds ANGPTL3 can be administered once daily, twice daily, once every two weeks, or once monthly. The duration of administration of the antibody that specifically binds ANGPTL3 can be dependent on the severity of an infection or the stage of sepsis a subject is afflicted with. For example, the antibody can be s administered for at least 1 or 2 weeks. Longer durations of administration are also contemplated.

The subject to whom the antibody that specifically binds ANGPTL3 is administered may have or be suspected of having bacteremia, endotoxemia, or viremia.

The methods described herein can comprise conjointly administering an additional therapeutic agent, for example, an antibody that specifically binds PCSK9.

The subject to whom the antibody that specifically binds ANGPTL3 is administered can be any subject susceptible to sepsis. Thus, in some embodiments, the subject is a mammal. In further embodiments, the mammal is human.

Pharmaceutical compositions are described that comprise an antibody that specifically binds ANGPTL3 and at least one additional therapeutic agent. In some embodiments, at least one additional therapeutic agent is an antibody that specifically binds PCSK9.

Methods to treat sepsis in a subject are provided that comprise administering the pharmaceutical composition comprising an antibody that specifically binds ANGPTL3 as described herein.

Methods are also provided for clearing microbial toxins complexed with a lipoprotein particle from blood, the methods comprising contacting the blood with an antibody that specifically binds ANGPTL3.

The microbial toxins that can be cleared using the antibodies comprise a bacterial, viral, or fungal toxin.

### DETAILED DESCRIPTION

The present disclosure relates to methods and compositions for treating sepsis, e.g., by neutralizing and/or clearing bacterial toxins bound to lipoprotein particles, thereby decreasing the amount of bacterial toxins that can elicit an immune reaction in a subject. In certain aspects, methods of treating sepsis are provided herein comprising administering an antibody that specifically binds to ANGPTL3 (e.g., evinacumab) to a subject having or suspected of having sepsis. In some embodiments, a treatment regimen includes an anti-ANGPTL3 antibody and at least one additional therapeutic (e.g., an additional antibody or antimicrobial). In some aspects, provided herein are pharmaceutical compositions comprising such antibodies, methods of treating a stage of sepsis, and methods of neutralizing and/or clearing microbial toxins.

### Definitions

For convenience, certain terms employed in the specification, examples, and appended claims are collected here.

The articles “a” and “an” are used herein to refer to one or to more than one (e.g., to at least one) of the grammatical object of the article. By way of example, “an element” means one element or more than one element.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

The term “binding,” “bound,” and “interacting” refer to an forming (or a formed) association between two molecules, e.g., between an antibody and target epitope, e.g., between an anti-ANGPTL3 antibody and ANGPTL3. In some embodiments, the association is a stable association. In some embodiments, the binding or interacting between two molecules that form an association is due to electrostatic, hydrophobic, ionic, and/or hydrogen-bond interactions under physiological conditions.

The term “lipoprotein” and “lipoprotein complexes” refer to any of the lipid-protein complexes in which lipids are transported in the blood. These particles often comprise a spherical hydrophobic core of triglycerides or esters (e.g., cholesteryl esters) surrounded by an amphipathic monolayer of phospholipids, cholesterol, and apolipoproteins.

The terms “polynucleotide” and “nucleic acid molecule” are used herein interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

The term “polypeptide” refers to a polymer of amino acids and its equivalent and does not refer to a specific length of the product; thus, peptides, oligopeptides and proteins are included within the definition of a polypeptide. This term also does not refer to, or exclude modifications of the polypeptide, for example, glycosylations, acetylations, phosphorylations, and the like. Included within the definition are, for example, polypeptides containing one or more analogs of an amino acid (including, for example, natural amino acids, etc.), polypeptides with substituted linkages as well as other modifications known in the art, both naturally and non-naturally occurring.

As used herein “systemic inflammatory response syndrome” or “SIRS” is defined as including both septic (i.e., sepsis or septic shock) and non-septic systemic inflammatory response (i.e., post-operative). “SIRS” is further defined according to the American College of Chest Physicians (ACCP) guidelines as the presence of two or more of A) temperature >38° C. or <36° C., B) heart rate >90 beats per minute, C) respiratory rate >20 breaths per minute, and D) white blood cell count >12,000 mm³ or <4,000 mm³.

“Sepsis” is a life-threatening condition caused by a dysregulated host response to infection. Sepsis is a syndrome that is typically identified by clinical symptoms exhibited by a subject. Sepsis is categorized into three stages: Systemic Inflammatory Response Syndrome (SIRS), severe sepsis, and septic shock.

As used herein, the term “subject” includes any human or non-human animal. For example, the methods and compositions described herein can be used to treat a subject having sepsis. In certain embodiments provided herein the subject is a human. The term “non-human animal” includes all vertebrates, e.g., mammals and non-mammals, such as non-human primates, sheep, dog, cow, chickens, amphibians, reptiles, etc.

The term “treating” includes prophylactic and/or therapeutic treatments. The term “prophylactic or therapeutic” treatment is art-recognized and includes administration to a subject of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

### Sepsis

Sepsis occurs when a response to a microbial infection becomes dysregulated, resulting in increased inflammation. As effector molecules and chemicals are deposited into the blood stream in response to infection, the resulting inflammation can become systemic. This inflammation can cause blood clots and block oxygen from reaching vital organs that can result in organ failure and/or death. Sepsis is categorized into three stages: Systemic Inflammatory Response Syndrome (SIRS), severe sepsis, and septic shock.

SIRS is denoted by a very high or low body temperature, high heart rate, high respiratory rate, high or low white blood cell count, and a known or suspected infection.

SIRS generally manifests as a combination of vital sign abnormalities including fever or hypothermia, tachycardia, tachypnea, and leukocytosis or leukopenia, and can be further characterized by two or more of the following variables:
Fever of more than 38° C. (100.4° F.) or less than 36° C. (96.8° F.)
Heart rate of more than 90 beats per minute (in absence of intrinsic heart disease)
Respiratory rate of more than 20 breaths per minute or arterial carbon dioxide tension (PaCO₂) of less than 32 mm Hg
Abnormal white blood cell count (>12,000/μL or <4,000/μL or >10% immature neutrophils
   SIRS is nonspecific and can be caused by ischemia, inflammation, trauma, burns, infection, pancreatitis, stress, organ injury, major surgery, fractures, or several insults combined. Thus, SIRS is not always related to infection. Clinically, SIRS/sepsis can include or be associated or complicated with hypotension, perfusion abnormalities, hypoperfusion, lacto-acidosis, pulmonary embolism, oliguria, organ dysfunction and/or end-organ failure.

The second stage of sepsis is severe sepsis. Severe sepsis is diagnosed when acute organ dysfunction begins or when sepsis and hypotension (low blood pressure) or hypoperfusion (decreased blood flow through an organ) are comorbidities. Organ dysfunction is characterized by symptoms such as decreased urine output, sudden changes in mental state, decreased blood platelet count, difficulty breathing, abnormal heart pumping function, and abdominal pain. Urine output is one of the factors measured immediately after diagnosis of sepsis to track progression of the condition.

Septic shock is the most severe stage of sepsis. It is characterized by the presence of hypotension, induced by sepsis, despite fluid resuscitation. Severe sepsis can be accompanied by the failure, or indications of failure, of at least one organ. Cardiovascular failure resulting from severe sepsis can also present with hypotension, respiratory failure due to hypoxemia, renal failure due to oliguria, and hematologic failure due to coagulopathy. Septic shock has the highest chance of mortality, with estimates ranging from 30% to 50%.

The immune response to the microbe is central to the development of septic shock. This immune response is often initiated by innate immune cells system (i.e., macrophages, neutrophils, NK cells) recognizing microbial products through a set of receptors known as pattern recognition receptors (PRRs) that can recognize a pathogen-associated molecular patterns or a micro-associated molecular pattern (such as lipopolysaccharide, LPS). For instance, toll-like receptor (TLR) activation by LPS triggers intercellular signaling and activation of transcriptional factors, such as NFkβ, which in turn activates proinflammatory cytokines, chemokines, coagulations factors, and proteases. The cellular redox state can dramatically influence the innate immune response. Patients suffering from late stage sepsis consistently show a decline in their immune responsiveness (immune deficiency of the adaptive immune system). In particular, the immune responsiveness of a patient is categorized by leukocytes producing increased levels of anti-inflammatory cytokines such as IL-10 and T cell anergy that can be associated with a shift in the Th cell pattern to a predominant Th2 response.

Risk factors for developing sepsis include, for example, higher age (e.g., 60-80 years or >80 years), renal insufficiency (e.g., chronic or acute renal failure or nephropathy), wound healing disturbances, and/or diabetes mellitus, particularly diabetes mellitus associated with diabetic foot or ulcer or diabetic wound infection. Further, the frequency of infections, such as urinary tract infections, respiratory infections, wound infections, gastrointestinal infections, cholecystitis, necrotizing fasciitis, foot ulcers, AIDS, and hepatitis are typically higher in diabetic patients than non-diabetic patients, and the increased frequency of infection increases the risk of sepsis.

Methods are provided herein for preventing or treating SIRS, severe sepsis, and septic shock. Methods are also provided for preventing organ tissue damage in the subject. In some embodiments, the organ tissue is liver tissue. In some embodiments, the organ tissue is kidney tissue. In some embodiments, the organ tissue is central nervous system tissue.

Sepsis can result from a dysregulated immune response to a pathogen in the blood. For example, bacteremia is the presence of viable bacteria in the bloodstream, and viremia and fungemia refer to viruses and fungi, respectively, in the bloodstream.

A non-exclusive list of sites of infection and microorganisms that cause sepsis is provided in Table 3.

TABLE 3
Common sites of infection and causative microorganisms of sepsis

| Common infection | |
|---|---|
| site | Common microorganisms |
| Pulmonary | Pneumococcus, staphylococcus, atypical infections, |
| | such as mycoplasma and legionella, viruses and |
| | Gram negatives |
| Abdominal | Gram negatives such as Escherichia coli, |
| | Klebsiella, anaerobic organisms, Enterococcus, |
| | Candida |
| Skin/soft tissue | Streptococcus, methicillin-sensitive Staphylococcus |
| | aureus, Gram negatives |
| Urinary | Gram-negative bacilli, Enterococcus |
| Intravascular | MRSA, Coagulase negative staphylococcus, Gram |
| catheters | negatives |
| Central nervous | Neisseria, pneumococcus, Gram positives |
| system | |
| Endocarditis | MSSA, coagulase negative staphylococcus |

### ANGPTL3

Bacterial toxins travel in the blood in or associated with lipoprotein particles and signal through Toll-like receptors (TLRs) on cells to cause downstream septic conditions (and septic shock). The invention described herein aims to remove the toxins from the blood quickly to prevent their effects on cells and tissues. ANGPTL3 inhibits lipoprotein lipase and endothelial lipase—both lipases promote clearance of lipoprotein particles (and toxins contained therein) from the blood. Therefore, inhibition of ANGPTL3 promotes the quick clearance of lipoprotein particles from the blood, thereby clearing the bacterial toxins in or associated with those particles from the blood.

ANGPTL3 is a member of the angiopoietin-like family of secreted factors. ANGPTL3 is expressed predominantly in the liver and has the characteristic structure of angiopoietins (a signal peptide, N-terminal coiled-coil domain, and the C-terminal fibrinogen (FBN)-like domain). Bacterial toxins released during a bacterial infection travel in the blood in lipoprotein particles and signal through toll like receptors (TLRs) on cells. Increasing concentrations of such lipoprotein particles comprising bacterial toxins can lead to sepsis progression in a subject. ANGPTL3 inhibits lipoprotein lipase and endothelial lipase, which both promote clearance of lipoprotein particles from the blood. Inhibiting the ability of ANGPTL3 to bind to or otherwise interact with these lipases by contacting ANGPTL3 with an antibody disclosed herein increases lipoprotein lipase and endothelial lipase activity and promotes clearance of the lipoprotein particles (and toxins contained therein) from the blood.

Exemplary ANGPTL3 polypeptide amino acid sequences are provided in Table 1.

TABLE 1
ANGPTL3 Polypeptide Amino Acid Sequences

| SEQ | | | | NCBI |
|---|---|---|---|---|
| ID | | | | Reference |
| NO. | Protein | Species | Amino Acid Sequence | Sequence |
| 1 | ANGPTL3 | H. sapiens | MFTIKLLLFIVPLVISSRIDQDNSSF | NP_055310 |
| | | | DSLSPEPKSRFAMLDDVKILANGL | |
| | | | LQLGHGLKDFVHKTKGQINDIFQ | |
| | | | KLNIFDQSFYDLSLQTSEIKEEEKE | |
| | | | LRRTTYKLQVKNEEVKNMSLELN | |
| | | | SKLESLLEEKILLQQKVKYLEEQL | |
| | | | TNLIQNQPETPEHPEVTSLKTFVE | |
| | | | KQDNSIKDLLQTVEDQYKQLNQQ | |
| | | | HSQIKEIENQLRRTSIQEPTEISLSS | |
| | | | KPRAPRTTPFLQLNEIRNVKHDGI | |
| | | | PAECTTIYNRGEHTSGMYAIRPSN | |
| | | | SQVFHVYCDVISGS | |
| | | | PWTLIQHRIDGSQNFNETWENYK | |
| | | | YGFGRLDGEFWLGLEKIYSIVKQS | |
| | | | NYVLRIELEDWKDNKHYIEYSFY | |
| | | | LGNHETNYTLHLVAITGNVPNAIP | |
| | | | ENKDLVFSTWDHKAKGHFNCPE | |
| | | | GYSGGWWWHDECGENNLNGKY | |
| | | | NKPRAKSKPERRRGLSWKSQNGR | |
| | | | LYSIKSTKMLIHPTDSESFE | |
| 2 | ANGPTL3 | Mus | MHTIKLFLFVVPLVIASRVDPDLS | NP_038941 |
| | | musculus | SFDSAPSEPKSRFAMLDDVKILAN | |
| | | | GLLQLGHGLKDFVHKTKGQIND | |
| | | | IFQKLNIFDQSFYDLSLRTNEIKEE | |
| | | | EKELRRTTSTLQVKNEEVKNMSV | |
| | | | ELNSKLESLLEEKTALQHKVRA | |
| | | | LEEQLTNLILSPAGAQEHPEVTSL | |
| | | | KSFVEQQDNSIRELLQSVEEQYKQ | |
| | | | LSQQHMQIKEIEKQLRKTGIQE | |
| | | | PSENSLSSKSRAPRTTPPLQLNETE | |
| | | | NTEQDDLPADCSAVYNRGEHTSG | |
| | | | VYTIKPRNSQGFNVYCDTQSGS | |
| | | | PWTLIQHRKDGSQDFNETWENYE | |
| | | | KGFGRLDGEFWLGLEKIYAIVQQ | |
| | | | SNYILRLELQDWKDSKHYVEYSFH | |
| | | | LGSHETNYTLHVAEIAGNIPGALP | |
| | | | EHTDLMFSTWNHRAKGQLYCPES | |
| | | | YSGGWWWNDICGENNLNGKYNKP | |
| | | | RTKSRPERRRGIYWRPQSRKLYAI | |
| | | | KSSKMMLQPTT | |
| 3 | ANGPTL3 | Rattus | MHTIKLLLFVVPLVISSRVDPDLSP | NP_001020236 |
| | | norvegicus | FDSVPSEPKSRFAMLDDVKILANG | |
| | | | LLQLGHGLKDFVHKTKGQIND | |
| | | | IFQKLNIFDQCFYDLSLQTNEIKEE | |
| | | | EKELRRTTSKLQVKNEEVKNMSL | |
| | | | ELNSKLESLLEEKMALQHRVRA | |
| | | | LEEQLTSLVQNPPGAREHPEVTSL | |
| | | | KSFVEQQDNSIRELLQSVEEQYKQ | |
| | | | LSQQHIQIKEIENQLRKTGIQEPTE | |
| | | | NSLYSKPRAPRTTPPLHLKEAKNI | |
| | | | EQDDLPADCSAIYNRGEHTSGVY | |
| | | | TIRPSSSQVFNVYCDTQSGT | |
| | | | PRTLIQHRKDGSQNFNQTWENYE | |
| | | | KGFGRLDGKVISLHHSLIC | |
| 4 | ANGPTL3 | Macaca | MFTIKLLLFIVPLVISSRIDQDNSSF | XP_014996673 |
| | | mulatta | DSVSPEPKSRFAMLDDVKILANG | |
| | | | LLQLGHGLKDFVHKTKGQINDIF | |
| | | | QKLNIFDQSFYDLSLQTSEIKEEEK | |
| | | | ELRRTTYKLQVKNEEVKNMSLEL | |
| | | | NSKLESLLEEKILLQQKVKYLEEQ | |
| | | | LTNLIQNQPETPEHPEVTSLKSFVE | |
| | | | KQDNSIKDLLQTVEEQYKQLNQQ | |
| | | | HSQIKEIENQLRMTNIQEPTEISLS | |
| | | | SKPRAPRTTPFLQLNEIRNVKHDG | |
| | | | IPADCTTIYNRGEHISGMYAIRPSN | |
| | | | SQVFHVYCDVVSGSPWTLIQHRI | |
| | | | DGSQNFNETWENYKYGFGRLDG | |
| | | | EFWLGLEKIYSIVKQSNYVLRIEL | |
| | | | EDWKDNKHYIEYSFYLGNHETNY | |
| | | | TLHVVKITGNVPNAIPENKDLVFS | |
| | | | TWDHKAKGHFSCPESYSGGWW | |
| | | | WHDECGENNLNGKYNKPRTKSK | |
| | | | PERRRGLSWKSQNGRLYSIKSTK | |
| | | | MLIHPTDSESFE | |
| 5 | ANGPTL3 | Pan | MFTIKLLLFIVPLVISSRIDQDNSSF | XP_001159650 |
| | | troglodytes | DSLSPEPKSRFAMLDDVKILANGL | |
| | | | LQLGHGLKDFVHKTKGQINDIFQ | |
| | | | KLNIFDQSFYDLSLQTSEIKEEEKE | |
| | | | LRRTTYKLQVKNEEVKNMSLELN | |
| | | | SKLESLLEEKILLQQKVKYLEEQL | |
| | | | TNLIQNQPETPEHPEVTSLKTFVE | |
| | | | KQDNSIKDLLQTVEDQYKQLNQQ | |
| | | | HSQIKEIENQLRRTSIQEPTEISLSS | |
| | | | KPRAPRTTPFLQLNEIRNVKRDGI | |
| | | | PAECTTIYNRGEHTSGMYAIRPSN | |
| | | | SQVFHVYCDVVSGSPWTLIQHRI | |
| | | | DGSQNFNETWENYKYGFGRLDG | |
| | | | EFWLGLEKIYSIVKQSNYVLRIEL | |
| | | | EDWKDNKHYIEYSFYLGNHETNY | |
| | | | TLHLVAITGNVPNAIPENKDLVFS | |
| | | | TWDHKAKGHFNCPEGYSGGWW | |
| | | | WHDECGENNLNGKYNKPRAKSK | |
| | | | PERRRGLSWKSQNGRLYSIKSTK | |
| | | | MLIHPTDSESFE | |

### Antibodies

Disclosed herein are antibodies that are effective therapeutic agents for treating sepsis. In particular, antibodies are disclosed that specifically bind to ANGPTL3. In some embodiments, the anti-ANGPTL3 antibody provided herein is administered conjointly with a PCSK9 inhibitor. In some embodiments, the anti-ANGPTL3 antibody is administered simultaneously or sequentially with a PCSK9 inhibitor. In some embodiments, the PCSK9 inhibitor is an antibody that specifically binds to PCSK9.

The term “antibody” as used to herein includes whole antibodies and any antigen binding fragments (i.e., “antigen-binding portions”) or single chains thereof. An “antibody” refers, in one embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. In certain naturally occurring antibodies, the heavy chain constant region is comprised of three domains, CH1, CH2 and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 5×10⁻⁹ M or less, and most preferably between 10⁻⁸ M and 10⁻¹⁰ M or less, but does not bind with high affinity to unrelated antigens. An antigen is “substantially identical” to a given antigen if it exhibits a high degree of sequence identity to the given antigen, for example, if it exhibits at least 80%, at least 90%, preferably at least 95%, more preferably at least 97%, or even more preferably at least 99% sequence identity to the sequence of the given antigen.

In some embodiments, the antibodies provided herein may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., IgG1, exist in several allotypes, which differ from each other in at most a few amino acids. “Antibody” includes, by way of example, both naturally occurring and non-naturally occurring antibodies; monoclonal and polyclonal antibodies; chimeric and humanized antibodies; human and nonhuman antibodies; wholly synthetic antibodies; and single chain antibodies.

In certain embodiments, provided herein are antigen-binding portions of antibodies. The term “antigen-binding portion” of an antibody, as used herein, refers to one or more fragments of an antibody that retain the ability to specifically bind to an antigen (e.g., ANGPTL3). Such “fragments” are, for example, between about 8 and about 1500 amino acids in length, between about 8 and about 745 amino acids in length, about 8 to about 300, about 8 to about 200 amino acids, or about 10 to about 50 or 100 amino acids in length. It has been shown that the antigen-binding function of an antibody can be performed by fragments of a full-length antibody. Examples of binding fragments encompassed within the term “antigen-binding portion” of an antibody, described herein, include an isolated complementarity determining region (CDR) or a combination of two or more isolated CDRs. Single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

In certain embodiments, the antibodies provided herein comprise one or more CDRs of antibodies. “CDRs” of an antibody are amino acid residues within the hypervariable region that are identified in accordance with the definitions of the Kabat, Chothia, AbM, contact, and/or conformational definitions or any method of CDR determination well known in the art. Antibody CDRs can be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., 1989, Nature 342:877-883. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., 1996, J. Mol. Biol., 262:732-745. In another approach, referred to herein as the “conformational definition” of CDRs, the positions of the CDRs can be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly affect antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

In some embodiments, the antibodies provided herein are monoclonal antibodies. The term “monoclonal antibody,” as used herein, refers to an antibody having a single binding specificity and affinity for a particular epitope or a composition of antibodies in which all antibodies display a single binding specificity and affinity for a particular epitope. Accordingly, the term “human monoclonal antibody” refers to an antibody or antibody composition that display(s) a single binding specificity and which has variable and optional constant regions derived from human germline immunoglobulin sequences. In one embodiment, human monoclonal antibodies are produced by a hybridoma, which comprises a B cell obtained from a transgenic non-human animal, e.g., a transgenic mouse having a genome comprising a human heavy chain transgene and a light chain transgene, fused to an immortalized cell.

In some embodiments, the antibodies provided herein are humanized antibodies. A “humanized” antibody refers to an antibody in which some, most, or all of the amino acids outside the CDR domains of a non-human antibody are replaced with corresponding amino acids derived from human immunoglobulins. In one embodiment of a humanized form of an antibody, some, most, or all of the amino acids outside the CDR domains have been replaced with amino acids from human immunoglobulins, whereas some, most, or all amino acids within one or more CDR regions are unchanged. Small additions, deletions, insertions, substitutions, or modifications of amino acids are permissible as long as they do not abrogate the ability of the antibody to bind to a particular antigen. A “humanized” antibody retains an antigenic specificity similar to that of the original antibody.

In certain embodiments, the antibodies provided herein are chimeric antibodies. A “chimeric antibody” refers to an antibody in which the variable regions are derived from one species and the constant regions are derived from another species, such as an antibody in which the variable regions are derived from a mouse antibody and the constant regions are derived from a human antibody.

In certain embodiments, the antibodies provided herein can be of any isotype. As used herein, “isotype” refers to the antibody class (e.g., IgG1, IgG2, IgG3, IgG4, IgM, IgA1, IgA2, IgD, and IgE) that is encoded by the heavy chain constant region genes. In some embodiments, the antibodies provided herein are IgG1, IgG2, IgG3, IgG4, IgM, IgA1, IgA2, IgD, or IgE isotype antibodies.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (Clq) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains Cγ2 and Cγ3 and the hinge between Cγ1 and Cγ2. As used herein, the Fc region may be a native sequence Fc, including any allotypic variant, or a variant Fc (e.g., a non-naturally occurring Fc). Fc may also refer to this region in isolation or in the context of an Fc-comprising protein polypeptide such as a “binding protein comprising an Fc region,” also referred to as an “Fc fusion protein” (e.g., an antibody or immunoadhesin).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc include the various allotypes of Fcs (see, e.g., Jefferis et al. (2009) mAbs 1:1).

A “hinge,” “hinge domain,” or “hinge region” or “antibody hinge region” refers to the domain of a heavy chain constant region that joins the CH1 domain to the CH2 domain and includes the upper, middle, and lower portions of the hinge (Roux et al. 1998 J. Immunol. 161:4083). The hinge provides varying levels of flexibility between the binding and effector regions of an antibody and provides sites for intermolecular disulfide bonding between the two heavy chain constant regions.

The term “hinge” includes wild type hinges as well as variants thereof (e.g., non-naturally-occurring hinges or modified hinges). The term “CH1 domain” refers to the heavy chain constant region linking the variable domain to the hinge in a heavy chain constant domain. The term “CH2 domain” refers to the heavy chain constant region linking the hinge to the CH3 domain in a heavy chain constant domain. The term “CH3 domain” refers to the heavy chain constant region that is C-terminal to the CH2 domain in a heavy chain constant domain.

The term “epitope” or “antigenic determinant” refers to a site on an antigen (e.g., ANGPTL3) to which an immunoglobulin or antibody specifically binds. Epitopes can be formed from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides (e.g., ANGPTL3) are tested for reactivity with a given antibody (e.g., anti-ANGPTL3 antibody). Methods of determining spatial conformation of epitopes include techniques in the art and those described herein, for example, x-ray crystallography, 2-dimensional nuclear magnetic resonance and HDX-MS (see, e.g., Epitope Mapping Protocols in Methods in Molecular Biology, Vol. 66, G. E. Morris, Ed. (1996)).

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE 2000 instrument using the predetermined antigen, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen.

In certain aspects, provided herein are nucleic acid molecules encoding an antibody provided herein. The term “nucleic acid molecule,” as used herein, is intended to include DNA molecules and RNA molecules. A nucleic acid molecule can be single-stranded or double-stranded. In some embodiments, a nucleic acid molecule is a cDNA molecule.

Also contemplated are “conservative sequence modifications” of the sequences set forth herein, e.g., in Tables 1 and 2, i.e., nucleotide and amino acid sequence modifications which do not abrogate the binding of the antibody encoded by the nucleotide sequence or containing the amino acid sequence, to the antigen. Such conservative sequence modifications include conservative nucleotide and amino acid substitutions, as well as, nucleotide and amino acid additions and deletions. For example, modifications can be introduced into a sequence by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-ANGPTL3 antibody is preferably replaced with another amino acid residue from the same side chain family. Methods of identifying nucleotide and amino acid conservative substitutions that do not eliminate antigen binding are well known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)).

In certain embodiments, provided herein are nucleic acid molecules having substantial homology to a sequence provided herein. For nucleic acid molecules, the term “substantial homology” indicates that two nucleic acid molecules, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

In certain embodiments, provided herein are antibodies having heavy and/or light chains with substantial homology to a sequence provided herein. For polypeptides, the term “substantial homology” indicates that two polypeptides, or designated sequences thereof, when optimally aligned and compared, are identical, with appropriate amino acid insertions or deletions, in at least about 80% of the amino acids, usually at least about 90% to 95%, and more preferably at least about 98% to 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available on the World Wide Web at gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

In some embodiments, provided herein are vectors encoding the heavy and/or light chain of an antibody provided herein. The term “vector,” as used herein, is intended to refer to a nucleic acid molecule capable of transporting another nucleic acid molecule to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

In some embodiments, provided herein is a host cell comprising a nucleic acid molecule disclosed herein. The term “recombinant host cell” (or simply “host cell”), as used herein, is intended to refer to a cell that comprises a nucleic acid molecule that is not naturally present in the cell, for example, a cell into which a recombinant expression vector has been introduced. It should be understood that such terms are intended to refer not only to the particular subject cell but also to the progeny of such a cell. Because certain modifications can occur in succeeding generations due to either mutation or environmental influences, such progeny may not, in fact, be identical to the parent cell, but are still included within the scope of the term “host cell” as used herein.

In certain aspects, provided herein are methods of neutralizing and/or clearing a microbial toxin, comprising contacting ANGPTL3 with an anti-ANGPTL3 monoclonal antibody. In some embodiments, the microbial toxin is a bacterial toxin. In some embodiments, the microbial toxin is a viral toxin.

Anti-ANGPTL3 antibodies are known in the art. Anti-ANGPTL3 antibodies and fragments thereof are described in U.S. Pat. No. 10,358,487, US 2017/0291937, US 2020/0079841, and WO2017189813, herein incorporated by reference in its entirety and in particular for the anti-ANGPTL3 antibodies disclosed therein.

In some embodiments, the anti-ANGPTL3 antibody is a monoclonal antibody. In some embodiments, the anti-ANGPTL3 monoclonal antibody disclosed herein is evinacumab. Evinacumab sequences are provided in WO2017189813, the disclosures of which are incorporated herein by reference, and in Table 5.

TABLE 5
Evinacumab Sequences

| SEQ | Sequence | |
|---|---|---|
| ID NO. | Region | Amino Acid Sequence |
| 11 | Heavy Chain | EVQLVESGGGVIQPGGSLRLSCAASG |
| | (HC) Variable | FTFDDYAMNWVRQGPGKGLEWVSAIS |
| | Region | GDGGSTYYADSVKGRFTISRDNSKNS |
| | | LYLQMNSLRAEDTAFFYCAKDLRNTI |
| | | FGVVIPDAFDIWGQGTMVTVSS |
| 12 | Light Chain | DIQMTQSPSTLSASVGDRVTITCRAS |
| | (LC) Variable | QSIRSWLAWYQQKPGKAPKLLIYKAS |
| | Region | SLESGVPSRFSGSGSGTEFTLTISSL |
| | | QPDDFATYYCQQYNSYSYTFGQGTKL |
| | | EIKR |
| 13 | HCDR1 | GFTFDDYA |
| 14 | HCDR2 | ISGDGGST |
| 15 | HCDR3 | AKDLRNTIFGVVIPDAFDI |
| 16 | LCDR1 | QSIRSW |
| 17 | LCDR2 | KAS |
| 18 | LCDR3 | QQYNSYSYT |

In some embodiments, the anti-ANGPTL3 monoclonal antibody is conjointly administered with a PCSK9 inhibitor. Some PCSK9 inhibitors are disclosed in EP 2,849,788, the disclosures of which are incorporated herein by reference. In some embodiments, the PCSK29 inhibitor is a monoclonal antibody that specifically binds to PCSK9. Sequences of anti-PCSK9 monoclonal antibodies are disclosed in U.S. Pat. No. 8,062,640, the disclosures of which are incorporated herein by reference.

### PCSK9

PCSK9 is a member of the peptidase S8 Family. The peptidase S8 Family includes proteases that process protein and peptide precursors trafficking through regulated or constitutive branches of the secretory pathway. The PCSK9 protein undergoes an autocatalytic processing event the ER and is constitutively secreted as an inactive protease into the extracellular matrix and trans-Golgi network. PCSK9 expressed in liver, intestine and kidney tissues and escorts specific receptors for lysosomal degradation. It plays a role in cholesterol and fatty acid metabolism. A non-exhaustive list of PCSK9 polypeptide amino acid sequences is provided in Table 2.

TABLE 3
PCSK9 Polypeptide Amino Acid Sequences

| SEQ | | | | NCBI |
|---|---|---|---|---|
| ID | | | | Reference |
| NO. | Protein | Species | Amino Acid Sequence | Sequence |
| 6 | PCSK9 | H. | MGTVSSRRSWWPLPLLLLLLLLLGP | NP_777596 |
| | | sapiens | AGARAQEDEDGDYEELVLALRSEE | |
| | | | DGLAEAPEHGTTATFHRCAKDPWR | |
| | | | LPGTYVVVLKEETHLSQSERTARRL | |
| | | | QAQAARRGYLTKILHVFHGLLPGF | |
| | | | LVKMSGDLLELALKLPHVDYIEEDS | |
| | | | SVFAQSIPWNLERITPPRYRADEYQ | |
| | | | PPDGGSLVEVYLLDTSIQSDHREIEG | |
| | | | RVMVTDFENVPEEDGTRFHRQASK | |
| | | | CDSHGTHLAGVVSGRDAGVAKGA | |
| | | | SMRSLRVLNCQGKGTVSGTLIGLEF | |
| | | | IRKSQLVQPVGPLVVLLPLAGGYSR | |
| | | | VLNAACQRLARAGVVLVTAAGNF | |
| | | | RDDACLYSPASAPEVITVGATNAQ | |
| | | | DQPVTLGT | |
| | | | LGTNFGRCVDLFAPGEDIIGASSDC | |
| | | | STCFVSQSGTSQAAAHVAGIAAMM | |
| | | | LSAEPELTLAELRQRLIHFSAKDVIN | |
| | | | EAWFPEDQRVLTPNLVAALPPSTH | |
| | | | GAGWQLFCRTVWSAHSGPTRMAT | |
| | | | AVARCAPDEELLSCSSFSRSGKRRG | |
| | | | ERMEAQGGKLVCRAHNAFGGEGV | |
| | | | YAIARCCLLPQANCSVHTAPPAEAS | |
| | | | MGTRVHCHQQGHVLTGCSSHWEV | |
| | | | EDLGTHKPPVLRPRGQPNQCVGHR | |
| | | | EASIHASCCHAPGLECKVKEHGIPA | |
| | | | PQEQVTVACEEGWTLTGCSALPGT | |
| | | | SHVLGAYAVDNTCVVRSRDVSTTG | |
| | | | STSEGAVTAVAICCRSRHLAQASQE | |
| | | | LQ | |
| 7 | PCSK9 | Mus | MGTHCSAWLRWPLLPLLPPLLLLL | NP_705793 |
| | | musculus | LLLCPTGAGAQDEDGDYEELMLAL | |
| | | | PSQEDGLADEAAHVATATFRRC | |
| | | | SKEAWRLPGTYIVVLMEETQRLQIE | |
| | | | QTAHRLQTRAARRGYVIKVLHIFY | |
| | | | DLFPGFLVKMSSDLLGLALKL | |
| | | | PHVEYIEEDSFVFAQSIPWNLERIIP | |
| | | | AWHQTEEDRSPDGSSQVEVYLLDT | |
| | | | SIQGAHREIEGRVTITDENS | |
| | | | VPEEDGTRFHRQASKCDSHGTHLA | |
| | | | GVVSGRDAGVAKGTSLHSLRVLNC | |
| | | | QGKGTVSGTLIGLEFIRKSQLI | |
| | | | QPSGPLVVLLPLAGGYSRILNAACR | |
| | | | HLARTGVVLVAAAGNFRDDACLY | |
| | | | SPASAPEVITVGATNAQDQPVT | |
| | | | LGTLGTNFGRCVDLFAPGKDIIGAS | |
| | | | SDCSTCFMSQSGTSQAAAHVAGIV | |
| | | | ARMLSREPTLTLAELRQRLIH | |
| | | | FSTKDVINMAWFPEDQQVLTPNLV | |
| | | | ATLPPSTHETGGQLLCRTVWSAHS | |
| | | | GPTRTATATARCAPEEELLSCS | |
| | | | SFSRSGRRRGDWIEAIGGQQVCKAL | |
| | | | NAFGGEGVYAVARCCLVPRANCSI | |
| | | | HNTPAARAGLETHVHCHQKDH | |
| | | | VLTGCSFHWEVEDLSVRRQPALRS | |
| | | | RRQPGQCVGHQAASVYASCCHAP | |
| | | | GLECKIKEHGISGPSEQVTVACE | |
| | | | AGWTLTGCNVLPGASLTLGAYSVD | |
| | | | NLCVARVHDTARADRTSGEATVA | |
| | | | AAICCRSRPSAKASWVQ | |
| 8 | PCSK9 | Rattus | MGIRCSTWLRWPLSPQLLLLLLLCP | NP_954862 |
| | | norvegicus | TGSRAQDEDGDYEELMLALPSQED | |
| | | | SLVDEASHVATATFRRCSKEA | |
| | | | WRLPGTYVVVLMEETQRLQVEQT | |
| | | | AHRLQTWAARRGYVIKVLHVFYD | |
| | | | LFPGFLVKMSSDLLGLALKLPHVE | |
| | | | YIEEDSLVFAQSIPWNLERIIPAWQQ | |
| | | | TEEDSSPDGSSQVEVYLLDTSIQSG | |
| | | | HREIEGRVTITDFNSVPEE | |
| | | | DGTRFHRQASKCDSHGTHLAGVVS | |
| | | | GRDAGVAKGTSLHSLRVLNCQGK | |
| | | | GTVSGTLIGLEFIRKSQLIQPSG | |
| | | | PLVVLLPLAGGYSRILNTACQRLAR | |
| | | | TGVVLVAAAGNFRDDACLYSPASA | |
| | | | PEVITVGATNAQDQPVTLGTL | |
| | | | GTNFGRCVDLFAPGKDIIGASSDCS | |
| | | | TCYMSQSGTSQAAAHVAGIVAMM | |
| | | | LNRDPALTLAELRQRLILFSTK | |
| | | | DVINMAWFPEDQRVLTPNRVATLP | |
| | | | PSTQETGGQLLCRTVWSAHSGPTR | |
| | | | TATATARCAPEEELLSCSSFSR | |
| | | | SGRRRGDRIEAIGGQQVCKALNAF | |
| | | | GGEGVYAVARCCLLPRVNCSIHNT | |
| | | | PAARAGPQTPVHCHQKDHVLTG | |
| | | | CSFHWEVENLRAQQQPLLRSRHQP | |
| | | | GQCVGHQEASVHASCCHAPGLECK | |
| | | | IKEHGIAGPAEQVTVACEAGWT | |
| | | | LTGCNVLPGASLPLGAYSVDNVCV | |
| | | | ARIRDAGRADRTSEEATVAAAICCR | |
| | | | SRPSAKASWVHQ | |
| 9 | PCSK9 | Macaca | MGTVSSRRSWWPLPLPLLLLLLLGP | NP_001106130 |
| | | mulatta | AGARAQEDEDGDYEELVLALRSEE | |
| | | | DGLADAPEHGATATFHRCAKD | |
| | | | PWRLPGTYVVVLKEETHRSQSERT | |
| | | | ARRLQAQAARRGYLTKILHVFHHL | |
| | | | LPGFLVKMSGDLLELALKLPHV | |
| | | | DYIEEDSSVFAQSIPWNLERITPARY | |
| | | | RADEYQPPKGGSLVEVYLLDTSIQS | |
| | | | DHREIEGRVMVTDFESVPE | |
| | | | EDGTRFHRQASKCDSHGTHLAGVV | |
| | | | SGRDAGVAKGAGLRSLRVLNCQG | |
| | | | KGTVSGTLIGLEFIRKSQLVQPV | |
| | | | GPLVVLLPLAGGYSRVFNAACQRL | |
| | | | ARAGVVLVTAAGNFRDDACLYSPA | |
| | | | SAPEVITVGATNAQDQPVTLGT | |
| | | | LGTNFGRCVDLFAPGEDIIGASSDC | |
| | | | STCFVSRSGTSQAAAHVAGIAAMM | |
| | | | LSAEPELTLAELRQRLIHFSA | |
| | | | KDVINEAWFPEDQRVLTPNLVAAL | |
| | | | PPSTHRAGWQLFCRTVWSAHSGPT | |
| | | | RMATAVARCAQDEELLSCSSFS | |
| | | | RSGKRRGERIEAQGGKRVCRAHNA | |
| | | | FGGEGVYAIARCCLLPQVNCSVHT | |
| | | | APPAGASMGTRVHCHQQGHVLT | |
| | | | GCSSHWEVEDLGTHKPPVLRPRGQ | |
| | | | PNQCVGHREASIHASCCHAPGLEC | |
| | | | KVKEHGIPAPQEQVIVACEDGW | |
| | | | TLTGCSPLPGTSHVLGAYAVDNTC | |
| | | | VVRSRDVSTTGSTSKEAVAAVAICC | |
| | | | RSRHLVQASQELQ | |
| 10 | PCSK9 | Pan | MGTVSSRRSWWPLPLLLLLLLLLGP | NP_001104592 |
| | | troglodytes | AGARAQEDEDGDYEELVLALRSEE | |
| | | | DGLAEAPEHGTTATFHRCAKD | |
| | | | PWRLPGTYVVVLKEETHLSQSERT | |
| | | | ARRLQAQAARRGYLTKILHVFHGL | |
| | | | LPGFLVKMSGDLLELALKLPHV | |
| | | | DYIEEDSSVFAQSIPWNLERITPPRY | |
| | | | RADEYQPPDGGSLVEVYLLDTSIQS | |
| | | | DHREIEGRVMVTDFENVPE | |
| | | | EDGTRFHRQASKCDSHGTHLAGVV | |
| | | | SGRDAGVAKGASMRSLRVLNCQG | |
| | | | KGTVSGTLIGLEFIRKSQLVQPV | |
| | | | GPLVVLLPLAGGYSRVLNAACQRL | |
| | | | ARAGVVLVTAAGNFRDDACLYSPA | |
| | | | SAPEVITVGATNAQDQPVTLGT | |
| | | | LGTNFGRCVDLFAPGEDIIGASSDC | |
| | | | STCFVSQSGTSQAAAHVAGIAAMM | |
| | | | LSAEPELTLAELRQRLIHFSA | |
| | | | KDVINEAWFPEDQRVLTPNLVAAL | |
| | | | PPSTHGAGWQLFCRTVWSAHSGPT | |
| | | | RMATAVARCAPDEELLSCSSFS | |
| | | | RSGKRRGERMEAQGGKLVCRAHN | |
| | | | AFGGEGVYAIARCCLLPQANCSIHT | |
| | | | APPAEAGMGTRVHCHQQGHVLT | |
| | | | GCSSHWEVEDLGTHKPPMLRPRGQ | |
| | | | PNQCVGHREASIHASCCRAPGLECK | |
| | | | VKEHGIPAPQEQVTVACEEGW | |
| | | | TLTGCSALPGTSHVLGAYAVDNTC | |
| | | | VVRSRDVSTAGSTSEEAVAAVAICC | |
| | | | RSRHLAQASQELQ | |

### PCSK9 Inhibitors

Exemplary PCSK9 inhibitors useful for the treatment of sepsis are described below and in US 2015/0140005, hereby incorporated by reference in its entirety and in particular for the inhibitors disclosed therein.

### Monoclonal Antibodies

Anti-PCSK9 antibodies are known in the art. Monoclonal antibodies (MAbs) that specifically bind to PCSK9 are capable of inhibiting PCSK9 activity. In some instances, the MAbs bind near the catalytic domain, which interacts with the low density lipoprotein receptor (LDLR) thereby inhibiting the catalytic activity of PCSK9 on LDLR. A number of these MAbs are in clinical trials (for example, AMG 145 (Amgen), 1D05-IgG2 (Merck & Co.), and SAR236553/REGN727/Alirocumab (Aventis/Regeneron)). Similarly, additional MAbs targeting PCSK9 are also in development (for example, RN-316 (Pfizer); LGT209 (Novartis); RG7652 (Roche/Genentech)). A number of PCSK9 inhibitory antibodies and fragments thereof are described in the patent literature as follows:
MERCK/SCHERING CORP. (PCT/US2008/081311);
SCHERING CORP. (PCT/US2011/056649);
REGENERON PHARMACEUTICALS, INC. (PCT/US2012/054756;
PCT/US2012/048574; PCT/US2009/068013);
SANOFI (PCT/EP2012/051318; PCT/EP2012/051320; PCT/EP2012/051321);
ELI LILLY AND COMPANY (PCT/US2012/054737);
AFFIRIS AG (PCT/EP2012/067950);
PFIZER (PCT/M2012/053534; PCT/IB2012/050924; PCT/M2010/053784);
NOVARTIS AG (PCT/EP2012/061045; PCT/US2012/041214;
PCT/EP2008/054417);
IRM LLC and NOVARTIS AG (PCT/US2012/024633; PCT/US2010/059959);
GENENTECH INC. and HOFFMANN LA ROCHE (PCT/US2011/024633);
MERCK SHARP & DOHME (PCT/US2010/054714; PCT/US2010/054640;
PCT/US2010/048849);
RINAT NEUROSCIENCE CORP/PFIZER (PCT/IB2009/053990);
MERCK & CO INC. (PCT/US2009/033369; PCT/US2009/033341;
PCT/US2007/023223; PCT/US2007/023213; PCT/US2007/023212;
PCT/US2007/023169); and
AMGEN INC. (PCT/US2008/074097),
   all of which are hereby incorporated by reference in their entirety and in particular for the anti-PCSK9 antibodies disclosed therein.

PCSK9-mediated activity on cell surface LDLRs has been reversed using antibodies that recognize epitopes on PCSK9. In particular, where those epitopes are associated with the catalytic domain. Intravenous infusion of an Amgen monoclonal antibody (AMG 145) specific for the catalytic domain of PCSK9 resulted in a significant reduction of circulating LDL-C levels as early as 8 hours after injection in non-human primates. Merck & Co.'s monoclonal antibody (1D05-IgG2) structurally mimics the EGFA domain of the LDLR. A single injection of 1D05-IgG2 was also found to antagonize PCSK9 function in non-human primates, resulting in reduced plasma LDL-C levels by up to 50%. Pfizer-Rinat and Sanofi-Aventis/Regeneron also have monoclonal antibodies (RN316 and SAR236553/REGN727, respectively), which are also in clinical trials.

### Peptides

Peptides that mimic the EGFA domain of the LDLR that binds to PCSK9 have been developed to inhibit PCSK9. Similarly, EGF-A peptides, fibronectin based scaffold domain proteins, which bind PCSK9, and neutralizing PCSK9 variants (for example, with a Pro/Cat domain), have been developed and all of which have been shown to inhibit PCSK9 activity. A number of PCSK9 inhibitory peptides are described in the patent literature as follows:
SCHERING CORP. (PCT/US2009/044883);
GENENTECH INC. and HOFFMANN LA ROCHE (PCT/US2012/043315);
SQUIBB BRISTOL MYERS CO. (PCT/US2011/032231; PCT/US2007/015298);
ANGELETTI P IST RICHERCHE BIO (PCT/EP2011/054646); and
AMGEN INC. (PCT/US2009/034775),
   all of which are hereby incorporated by reference in their entirety and in particular for the inhibitors disclosed therein.

### Oligonucleotides

A PCSK9 antisense oligonucleotide from Isis Pharmaceuticals/Bristol-Myers Squibb (BMSPCSK9Rx) has been shown to increase expression of the LDLR and decrease circulating total cholesterol levels in mice. Similarly, a locked nucleic acid from Santaris Pharma (LNA ASO) reduced PCSK9 mRNA levels in mice. LNA ASO is complementary to the human and mouse PCSK9 mRNA (accession #NMI 74936 and NM153565) is a 13-nucleotide long gapmer with the following sequence: GTctgtggaaGCG (uppercase LNA, lowercase DNA) and phos-phorothioate internucleoside linkages. Alnylam Pharmaceuticals has shown positive results in clinical trials for an siRNA (ALN-PCS) for the inhibition of PCSK9. The siRNA was incorporated into lipidoid nanoparticles to minimize toxicity and intravenously infused in rats, mice, and monkeys, resulting in reduced LDL-C levels after administration. A number of PCSK9 inhibitory oligonucleotides are described in the patent literature as follows:
SANTARIS PHARMA A/S (PCT/EP2007/060703; PCT/EP2009/054499;
PCT/EP20 I 0/059257);
ISIS PHARMACEUTICAL INC. (PCT/US2007/068404);
SIRNA THERAPEUTICS INC. (PCT/US2007/073723);
ALNYLAM PHARMACEUTICALS INC. (PCT/US2011/058682;
PCT/US2010/047726; PCT/US2010/038707; PCT/US2009/032743; PCT/US2007/068655);
RXI PHARMACEUTICALS CORP. (PCT/US2010/000019)
INTRADIGM CORP. (PCT/US2009/036550); and
NASTECH PHARM CO. (PCT/US2008/055554),
   all of which are hereby incorporated by reference in their entirety and in particular for the inhibitors disclosed therein

### Small Molecules

Serometrix has reported a small molecule inhibitor of PCSK9 (SX-PCSK9). Similarly, berberine as described in the examples may be used as a PCSK9 inhibitor. Additional small molecule inhibitors of PCSK9 are described in WO 2020/150473 and WO 2020/150474, both of which are hereby incorporated by reference in their entirety and in particular for the inhibitors disclosed herein.

### Pharmaceutical Compositions

In certain aspects, provided herein are pharmaceutical compositions comprising an antibody that specifically binds ANGPTL3 and at least one additional therapeutic agent. In some embodiments, the additional therapeutic agent is an antibody that specifically binds PCSK9.

In an embodiment, a pharmaceutical composition comprising an antibody that specifically binds ANGPTL3 and at least one additional therapeutic agent can be used to treat sepsis, including specific stages of sepsis. For example, in one embodiment, the pharmaceutical composition is for use in treating SIRS. In another embodiment, the pharmaceutical composition is for use in treating severe sepsis. In another embodiment, the pharmaceutical composition is for use in treating septic shock.

In certain embodiments, the compositions and methods provided herein can be utilized to treat a subject in need thereof. In certain embodiments, the subject is a mammal such as a human, or a non-human mammal. When administered to a subject, such as a human, the composition or the compound is preferably administered as a pharmaceutical composition comprising, for example, a therapeutic compound and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In certain embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

In certain embodiments, the pharmaceutical compositions provided herein comprise a pharmaceutically acceptable carrier. The phrase “pharmaceutically acceptable carrier” as used herein means a pharmaceutically acceptable material, composition or vehicle, such as a liquid or solid filler, diluent, excipient, solvent or encapsulating material. A pharmaceutically acceptable carrier can contain physiologically acceptable agents that act, for example, to stabilize, increase solubility or to increase the absorption of a compound. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a therapeutic compound. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In certain embodiments, the pharmaceutical compositions provided herein can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973; 5,763,493; 5,731,000; 5,541,231; 5,427,798; 5,358,970; and 4,172,896, as well as in patents cited therein.

The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound with the carrier and, optionally, one or more accessory ingredients. In general, the formulations are prepared by uniformly and intimately bringing into association a compound with liquid carriers, or finely divided solid carriers, or both, and then, if necessary, shaping the product.

Formulations suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include adjuvants such as wetting agents, emulsifying and suspending agents, sweetening, flavoring, coloring, perfuming, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

In certain embodiments, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions can be varied to obtain an amount of the active ingredient that is effective to achieve the desired therapeutic response for a particular subject, composition, and mode of administration, without being toxic to the subject.

If desired, the effective daily dose of the active compound can be administered as one, two, three, four, five, six or more sub-doses administered separately at appropriate intervals throughout the day, optionally, in unit dosage forms. In certain embodiments, the active compound is administered two or three times daily. In some embodiments, the active compound is administered once daily.

In certain embodiments, compounds can be used alone or conjointly administered with another type of therapeutic agent (e.g., an antiviral agent). As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of therapeutic compounds with one or more additional therapeutic agent(s) (e.g., one or more additional chemotherapeutic agent(s)) provides improved efficacy relative to each individual administration of the compound (e.g., copper ionophore) or the one or more additional therapeutic agent(s). In certain such embodiments, the conjoint administration provides an additive effect, wherein an additive effect refers to the sum of each of the effects of individual administration of the therapeutic compound and the one or more additional therapeutic agent(s).

Pharmaceutically acceptable salts of compounds in the methods provided herein. In certain embodiments, contemplated salts include, but are not limited to, alkyl, dialkyl, trialkyl or tetra-alkyl ammonium salts. In certain embodiments, contemplated salts include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts include, but are not limited to, Na, Ca, K, Mg, Zn, copper, cobalt, cadmium, manganese, or other metal salts.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Methods of Treatment

In certain aspects, provided herein are methods of preventing or treating sepsis in a subject, comprising administering a therapeutically effective dose of an anti-ANGPTL3 antibody that specifically binds to ANGPTL3 to a subject. At a molecular level, methods are presented for neutralizing and/or clearing a microbial toxin that is bound or otherwise associated with lipoprotein particles and lipids in the blood. Clearance of these toxin-lipoprotein complexes from the blood is promoted by the activity of lipoprotein lipase and endothelial lipase. ANGPTL3 inhibits these lipases, thereby reducing clearance of the bacterial toxin in the blood, which can then accumulate and lead to or exacerbate sepsis. Inhibiting the ability of ANGPTL3 neutralize or otherwise reduce the activity of lipoprotein lipase and endothelial lipase by contacting ANGPTL3 with an antibody as disclosed herein increases lipoprotein lipase and endothelial lipase activity, which in turn facilitates processing and clearance of these lipoprotein-toxin complexes (and toxins contain therein) from the blood.

In certain embodiments, the subject being treated for sepsis is administered an effective dose of an anti-ANGPTL3 antibody. The term “effective dose” or “effective dosage” refers to an amount sufficient to achieve or at least partially achieve a desired effect (i.e., a reduction in at least one symptom associated with sepsis). A “therapeutically effective amount” or “therapeutically effective dosage” of a therapeutic agent is any amount of the agent that, when used alone or in combination with another therapeutic agent, promotes regression of sepsis as evidenced by a decrease in severity of symptoms, an increase in frequency and duration of symptom-free periods, or a prevention of impairment or disability due to sepsis. A therapeutically effective amount or dosage of an agent includes a “prophylactically effective amount” or a “prophylactically effective dosage,” which is any amount of the agent that, when administered alone or in combination with another therapeutic agent to a subject at risk of developing sepsis or of suffering a recurrence of sepsis, inhibits the development or recurrence of sepsis. The ability of a therapeutic agent to promote regression of sepsis or inhibit the development or recurrence of the condition can be evaluated using a variety of methods known to the skilled practitioner, such as in human subjects during clinical trials, in animal model systems predictive of efficacy in humans, or by assaying the activity of the agent in in vitro assays.

In one aspect, a method is provided for modulating an immune response in a subject having or suspected of having sepsis, the method comprising administering to the subject a therapeutically effective dose of an antibody that specifically binds ANGPTL3. In one embodiment, modulation of an immune response can be detected by measuring a change in inflammation in a subject. Methods of characterizing or measuring inflammation in a subject are known in the art. For example, changes inflammation can be measured or characterized by detecting changes in the amount or a level of a biomarker associated with inflammation. Such biomarkers include, but are not limited to, cytokines/chemokines, immune-related effectors, acute phase proteins (C-reactive protein, Serum Amyloid A), reactive oxygen and nitrogen species, prostaglandins and cyclooxygenase-related factors, and mediators such as transcription factors and growth factors. Erythrocyte sedimentation rate (ESR) and plasma viscosity (PV) are common methods for evaluating inflammatory conditions.

In some embodiments, the method comprises administering an anti-ANGPTL3 antibody conjointly with an additional sepsis therapeutic agent. In some embodiments, the additional sepsis therapeutic agent is an antibiotic. Exemplary antibiotics for treatment of sepsis include but are not limited to ceftriaxone (Rocephin), meropenem (Merrem), ceftazidime (Fortaz), cefotaxime (Claforan), cefepime (Maxipime), piperacillin and tazobactam (Zosyn), ampicillin and sulbactam (Unasyn), imipenem/cilastatin (Primaxin), levofloxacin (Levaquin), or clindamycin (Cleocin). In some embodiments, the additional sepsis agent prevents, reduces, or otherwise modifies an inflammatory response and/or protects the organs and/or tissues of a subject. In some embodiments, the additional sepsis therapeutic agent is a steroid or corticosteroid. In some embodiments, the additional sepsis therapeutic agent is a vasopressor. In some embodiments, the additional sepsis therapeutic agent is an inotrope. In some embodiments, the additional sepsis therapeutic agent is a glucocorticoid.

In some embodiments, the effective dose is at least 5 mg, 10 mg, 15 mg, 20 mg, 25 mg, 50 mg, 100 mg, 150 mg, 200 mg, 250 mg, 300 mg, 350 mg, 400 mg, or 450 mg. In some embodiments, the effective dose is at most 5 mg, 25 mg, 50 mg, 100 mg, 150 mg, 200 mg, 250 mg, 300 mg, 350 mg, 400 mg, or 450 mg. In some embodiments, the effective dose is about 5 mg, 25 mg, 50 mg, 100 mg, 150 mg, 200 mg, 250 mg, 300 mg, 350 mg, 400 mg, or 450 mg. For example, in some embodiments, the effective dose is between about 5 mg and about 20 mg when administered intravenously. In other embodiments, the effective dose is between about 50 mg and 150 mg when administered subcutaneously.

In some embodiments, the anti-ANGPTL3 antibody is administered once daily, once weekly, or once monthly. In other embodiments, the anti-ANGPTL3 antibody is administered twice daily. In still other embodiments, the anti-ANGPTL3 antibody is administered once weekly or once monthly.

In some embodiments, the anti-ANGPTL3 antibody is administered for at least 1 week. In some embodiments, the anti-ANGPTL3 antibody is administered for at least 2 weeks.

### Incorporation by Reference

All publications, patents, and patent applications mentioned herein are hereby incorporated by reference in their entirety as if each individual publication, patent, or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A method of preventing or treating sepsis in a subject, the method comprising administering to the subject an antibody that specifically binds ANGPTL3.

2. A method of preventing or treating organ tissue damage in a subject, the method comprising administering to the subject an antibody that specifically binds ANGPTL3.

3. The method of claim 2, wherein the organ tissue is liver tissue.

4. The method of claim 2, wherein the organ tissue is kidney tissue.

5. The method of claim 2, wherein the organ tissue is central nervous system tissue.

6. A method of modulating an immune response in a subject having or suspected of having sepsis, the method comprising administering to the subject an antibody that specifically binds ANGPTL3.

7. The method of any one of claims 1-6, wherein the sepsis is systemic inflammatory response syndrome (SIRS), severe sepsis, or septic shock.

8. The method of any one claims 1-7, wherein the antibody that specifically binds ANGPTL3 is a monoclonal antibody.

9. The method of any one claims 1-7, wherein the antibody that specifically binds ANGPTL3 is a polyclonal antibody.

10. The method of any one of claims 1-9, wherein the antibody that specifically binds ANGPTL3 is a chimeric antibody.

11. The method of any one of claims 1-10, wherein the antibody that specifically binds ANGPTL3 is a humanized antibody.

12. The method of any one of claims 1-8, wherein the antibody that specifically binds ANGPTL3 is evinacumab.

13. The method of any one of claims 1-12, wherein the therapeutically effective dose is between about 5 mg and about 450 mg.

14. The method of any one of claims 1-13, wherein the antibody that specifically binds ANGPTL3 is administered intravenously, intraperitoneally, intramuscularly, intraarterially, intraportally, intralesionally, orally, or subcutaneously.

15. The method of any one of claims 1-14, wherein the antibody that specifically binds ANGPTL3 is administered once daily, twice daily, once every two weeks, or once monthly.

16. The method of any one of claims 1-14, wherein the antibody that specifically binds ANGPTL3 is administered continuously via intravenous infusion.

17. The method of any one of claims 1-14, wherein the antibody that specifically binds ANGPTL3 is administered for at least 1 week.

18. The method of any one of claims 1-14, wherein the antibody that specifically binds ANGPTL3 is administered for at least 2 weeks.

19. The method of any one of claims 1-18, wherein the subject has or is suspected of having bacteremia.

20. The method of any one of claims 1-18, wherein the subject has or is suspected of having endotoxemia.

21. The method of any one of claims 1-18, wherein subject has or is suspected of having viremia.

22. The method of any one of claims 1-21, wherein the method further comprises conjointly administering an additional therapeutic agent.

23. The method of claim 22, wherein the additional therapeutic agent is an antibody that specifically binds PCSK9.

24. The method of any one of claims 1-23, wherein the subject is a mammal.

25. The method of claim 24, wherein the mammal is a human.

26. A pharmaceutical composition comprising an antibody that specifically binds ANGPTL3 and at least one additional therapeutic agent.

27. The pharmaceutical composition of claim 26, wherein at least one additional therapeutic agent is an antibody that specifically binds PCSK9.

28. A method of treating sepsis in a subject comprising administering the pharmaceutical composition of claim 26 or 27.

29. The method of claim 28, wherein the subject is a mammal.

30. The method of claim 29, wherein the mammal is a human.

31. A method of clearing microbial toxins complexed with a lipoprotein particle from blood, the method comprising contacting the blood with an antibody that specifically binds ANGPTL3.

32. The method of claim 31, wherein the antibody that specifically binds ANGPTL3 is a monoclonal antibody.

33. The method claim 31, wherein the antibody that specifically binds ANGPTL3 is a polyclonal antibody.

34. The method of any one of claims 31-33, wherein the antibody that specifically binds ANGPTL3 is a chimeric antibody.

35. The method of any one of claims 31-34, wherein the antibody that specifically binds ANGPTL3 is a humanized antibody.

36. The method of claim 31 or 32, wherein the antibody that specifically binds ANGPTL3 is evinacumab.

37. The method of any one of claims 31-36, wherein the microbial toxins comprise a bacterial toxin.

38. The method of any one of claims 31-36, wherein the microbial toxins comprise a viral toxin.

39. The method of any one of claims 31-36, wherein the microbial toxins comprise a fungal toxin.
